(19) United States
(12) Patent Application Publication
(10) Pub. No.: US 2008/0241899 A1
(43) Pub. Date: Oct. 2, 2008

(54) Poly (3-Hydroxyalkanoate) Block Copolymer Having Shape Memory Effect

(75) Inventors: Young Ha Rhee, Daejeon (KR); Young baek Kim, Daejeon (KR); Chungwook Chung, Daejeon (KR); Jooseog Yoon, Daejeon (KR)

Correspondence Address: CANTOR COLBURN, LLP, 20 Church Street, 22nd Floor, Hartford, CT, 06103, US

(73) Assignee: CHABIOTECH CO., LTD, SEOUL (KR)

(21) Appl. No.: 10/583,840

(22) PCT Filed: Dec. 22, 2005
(86) PCT No.: PCT/KR05/04439

§ 371 Date: Mar. 30, 2007

(30) Foreign Application Priority Data
Jul. 4, 2005 (KR) 10-2005-0059907

(51) Int. Cl. C12P 7/42; C07H 21/04; C08L 61/02

(52) U.S. Cl. 435/146; 536/23.1; 525/471

(57) ABSTRACT

Provided is a PHB block copolymer having orientation-induced rubber-elasticity and temperature-sensitive shape memory effects, comprising a plurality of 3-hydroxybutyrate (3HB) blocks as a repeating unit and a plurality of 3-hydroxyvalerate (3HV) blocks as a repeating unit, and optionally a hydroxy acid repeating group containing 6 or more carbon atoms.

The PHA block copolymer exhibits orientation-induced rubber-elasticity and shape memory effects with a fast shape-recovery rate, and therefore such characteristics in conjunction with physical properties such as biodegradability and biocompatibility unique to PHA enable application thereof to a variety of uses.

### FIELD OF THE INVENTION

The present invention relates to a poly(3-hydroxyalkanoate) block copolymer having shape memory effects. More specifically, the present invention relates to a block copolymer comprising a 3-hydroxybutyrate block as a repeating unit and a 3-hydroxyvalerate block as a repeating unit, and optionally comprising a hydroxy acid repeating group containing 6 or more carbon atoms, whereby the copolymer has orientation-induced rubber-elasticity and temperature-sensitive shape memory effects.

### BACKGROUND OF THE INVENTION

Poly(3-hydroxyalkanoates) (hereinafter, often referred to as “PHAs”) are polymers having superior mechanical properties as well as unique properties such as biodegradability and biocompatibility, and therefore a great deal of research and study has been made thereon. PHAs are generally classified into short-chain-length PHAs (SCL-PHAs), medium-chain-length PHAs (MCL-PHAs) and long-chain-length PHAs (LCL-PHAs), depending upon the number of carbon atoms of the constituting monomers thereof.

SCL-PHAs are PHAs in which the number of carbon atoms of the monomers constituting PHAs, such as 3-hydroxybutyrate (hereinafter, often referred to as “3HB”), 3-hydroxyvalerate (hereinafter, often referred to as “3HV”) and 4-hydroxybutyrate, is not more than 5, and include, for example a poly(3-hydroxybutyrate) (PHB) homopolymer and poly(3-hydroxybutyrate-co-3-hydroxyvalerate) (hereinafter, referred to as poly(3HB-co-3HV)) copolymer. MCL-PHAs are PHAs consisting of monomers containing 6 to 12 carbon atoms such as 3-hydroxyhexanoate (3HHx), 3-hydroxyheptanoate (3HHp) and 3-hydroxyoctanoate (3HO), and include, for example homopolymers or copolymers of such monomers. Finally, LCL-PHAs are PHAs consisting of monomers containing 13 or more carbon atoms, and include, for example homopolymers or copolymers of such monomers.

These PHAs may be synthesized by chemical synthesis or biosynthesis. In particular, methods for preparing PHAs via biosynthesis using microorganisms are well known in the art. Hitherto, microorganisms including more than 90 genera are known to biosynthesize PHAs. In addition, it is also known that there are more than 150 kinds of monomers of PHAs that are prepared via biosynthesis.

PHAs exhibit various physical properties depending upon kinds and compositions of monomers. Further, due to their diversity, it is believed that there are numerous physical properties that have yet to be identified.

### SUMMARY OF THE INVENTION

Therefore, the present invention has been made in view of the above problems, and it is an object of the present invention to provide novel physical properties of a PHA block copolymer, a method for preparing such a PHA block copolymer and use thereof.

As a result of a variety of extensive and intensive studies and experiments, the inventors of the present invention have surprisingly discovered that a PHA block copolymer having a specific composition exhibits shape memory effects. Shape memory effects are physical properties that were partially confirmed in metals and general polymers, but were not confirmed hitherto in PHAs primarily prepared via biosynthesis. In particular, it was confirmed that a certain PHA block copolymer provided herein exhibits orientation-induced rubber-elasticity in conjunction with temperature-sensitive shape memory effects and has fast shape-recovery ability. Such characteristics in conjunction with physical properties such as biodegradability and biocompatibility intrinsic to PHAs offer opportunities that enable PHAs to be utilized in a variety of applications. The present invention has been completed based on these findings. In accordance with an aspect of the present invention, the above and other objects can be accomplished by the provision of a PHB block copolymer having orientation-induced rubber-elasticity and temperature-sensitive shape memory effects, comprising:

a plurality of 3-hydroxybutyrate (3HB) blocks represented by Formula 1 as shown below as a repeating unit:

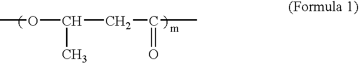

wherein m is not less than 2; and

a plurality of 3-hydroxyvalerate (3HV) blocks represented by Formula 2 as shown below as a repeating unit:

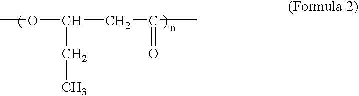

wherein n is not less than 2; and

optionally a plurality of hydroxy acid blocks containing 6 or more carbon atoms, represented by Formula 3 as shown below:

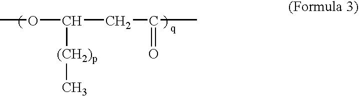

wherein p and q are independently not less than 2.

That is, the poly(3HB-co-3HV) block copolymer in accordance with the present invention can impart a temporary shape with rubber-elasticity and exerts temperature-sensitive shape memory effects. Herein, although the block copolymer in accordance with the present invention also encompasses a block copolymer in the form of poly(3HB-co-3HV-co-HA), poly(3HB-co-3HV) will be representatively illustrated hereinafter. It is speculated that action mechanisms for such characteristics are caused by induced orientation of soft segments and hard segments, which are formed by a plurality of 3HB blocks and 3HV blocks contained in one polymer molecule, resulting from application of external force and changes in temperatures. Hard segments prevent permanent deformation of the shaped materials. Chain entanglement and physical crosslinkers such as blocks of molecules exhibiting higher glass transition temperatures and melting points serve as hard segments. Soft segments are blocks of molecules having lower glass transition temperatures, and can induce deformation, thus making it possible to provide reversible setting and releasing properties.

Based on such unique physical properties, the poly(3HB-co-3HV) block copolymer in accordance with the present invention can be heated to a temperature ranging from a melting point to thermal decomposition temperature thereof, thus making it possible to prepare a permanently deformed particular shape. In addition, it is possible to obtain a shaped material having a temporary shape by applying constant external force to the permanently shaped material, for example at room temperature for a predetermined period of time. Such a temporarily shaped material is rapidly recovered to its original state of the permanently shaped material when it is heated to a temperature ranging from a glass transition temperature to melting point thereof.

In particular, shape memory effects are completely novel physical properties which were not known hitherto in PHAs and thus are highly significant in that applicability of PHAs to various fields can be greatly extended. Further, according to experiments of the present inventors, it was confirmed that a recovery rate in terms of shape memory effects is very fast, and such a shape recovery rate is higher than those of other general synthetic polymers.

Further, in most cases of conventional shape memory polymers, in order to set shapes thereof, a sample is heated to a temperature above a glass transition temperature of the soft segment, followed by deformation, and the temperature of the sample is then lowered below the glass transition temperature while keeping a deformed state, thereby maintaining a temporary shape thereof. Whereas, even when it is stretched at a temperature below the glass transition temperature, the shape memory polymer in accordance with the present invention can maintain a deformed shape by means of orientation induced crystallization and thereby can provide convenience in terms of processing.

The numbers of 3HB blocks and 3HV blocks present in one polymer molecule are not particularly limited so long as the numbers of blocks are within the range such that orientation-induced rubber-elasticity and shape memory effects are exerted while permitting to take a form of a block copolymer. For example, a content of 3HV in the total monomers of the copolymer is preferably within a range of 10 to 90 mol %, more preferably 20 to 80 mol %, and particularly preferably 30 to 70 mol %.

A molecular weight of the poly(3HB-co-3HV) block copolymer in accordance with the present invention is approximately in a range of several tens of thousands to several millions, preferably several hundreds of thousands, and more specifically in a range of 300,000 to 600,000.

If necessary, the PHA block copolymer in accordance with the present invention, as described above, may further comprise not more than 70 mol %, preferably not more than 50 mol %, and more preferably not more than 30 mol % of the hydroxy alkanoate (HA) block of Formula 3 as shown below, based on the total polymer:

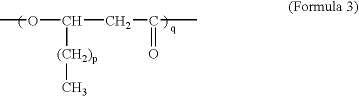

wherein p and q are independently not less than 2.

In accordance with another aspect of the present invention, there is provided a method for preparing the above-mentioned poly(3HB-co-3HV) block copolymer having shape memory effects. The poly(3HB-co-3HV) block copolymer can be prepared chemical synthesis, or biosynthesis using microorganisms. The latter is particularly preferred. As to biosynthesis of the poly(3HB-co-3HV) block copolymer using microorganisms, the inventors of the present invention have provided a Pseudomonas sp. HJ-2 strain (hereinafter, referred to as “HJ-2”) in Korean Patent Publication Laid-open No. 1999-0080695, which was deposited with the Korean Collection for Type Cultures (KCTC) affiliated with the Korean Research Institute of Bioscience and Biotechnology (KRIBB, Korea), under Accession Number KCTC 0406 BP. When it is cultured in a culture medium containing saturated and/or unsaturated carboxylic acids and/or carbohydrates such as glucose, starch or the like, the HJ-2 strain produces PHA copolymers containing various monomers. Therefore, although the disclosure of Korean Patent Publication Laid-open No. 1999-0080695 is incorporated by reference herein in its entirety, it should be construed that utilization of any other strains capable of synthesizing the shape-memory poly(3HB-co-3HV) block copolymer in accordance with the present invention other than the HJ-2 strain falls within the scope of the present invention.

In one preferred embodiment, it is possible to prepare the shape-memory poly(3HB-co-3HV) block copolymer in accordance with the present invention in a relatively high concentration by culturing the HJ-2 strain with supply of heptanoic acid as a sole carbon source.

The HJ-2 strain harbors both a short-chain-length PHA synthetic gene and a long-chain-length PHA synthetic gene, and the shape-memory poly(3HB-co-3HV) block copolymer can be biosynthesized by the short-chain-length PHA synthetic gene. Therefore, the present invention provides the short-chain-length PHA synthetic gene of the HJ-2 strain that is capable of biosynthesizing the poly(3HB-co-3HV) block copolymer having shape-memory effects.

Preferably, the above short-chain-length PHA synthetic gene is a gene including a gene having a sequence as set forth in SEQ. ID. NO: 12, a gene having a sequence as set forth in SEQ. ID. NO: 13, and/or a gene having a sequence as set forth in SEQ. ID. NO: 14.

Further, the shape-memory poly(3HB-co-3HV) block copolymer in accordance with the present invention may be synthesized by culturing a microorganism transformed with the short-chain-length PHA synthetic gene of the HJ-2 strain or by cell-free protein synthesis using the above-mentioned gene.

In accordance with a further aspect of the present invention, there is provided a method for application of a shape-memory poly(3HB-co-3HV) block copolymer to various uses.

In accordance with yet another aspect of the present invention, there is provided a blending or composite comprising a shape-memory poly(3HB-co-3HV) or poly(3HB-co-3HV-co-HA) block copolymer and a method for application thereof to various uses.

Surprisingly, it was further confirmed that a blending or composite, in which the shape-memory poly(3HB-co-3HV) or poly(3HB-co-3HV-co-HA) block copolymer is mixed with a general-purpose polymer resin such as polyvinylchloride (PVC), also exhibits shape memory effects. Therefore, in the case of PVC essentially requiring addition of a plasticizer in terms of manufacturing processes or uses thereof, it is possible to avoid use of the plasticizer which has recently become susceptible to control and regulation associated with use thereof due to possible generation of carcinogenic substances, by preparing PVC in the form of the above blending or composite material. However, application examples as mentioned above are only illustrative and therefore it should be understood that more and broader application examples are possible and are all encompassed within the scope of the present invention.

Representative examples of uses to which the shape-memory poly(3HB-co-3HV) block copolymer can be applied may include medical materials, materials for living necessaries, fiber/fabric materials, industrial materials and the like. In addition to shape-memory effects, the poly(3HB-co-3HV) block copolymer also possesses biodegradability, biocompatibility and superior mechanical properties, and can thus be particularly preferably used as medical materials.

The medical materials may include, but are not limited to, for example angioplasty stents, implant tubes for the urethrae and the esophagi, devices for vascular anastomosis, dental implants, and orthodontic springs or wires.

The materials for living necessaries may include, but are not limited to, for example cosmetics, massage packs, shape memory matrices, packaging materials or packaging films, and contraceptive devices.

The fiber/fabric materials may include, but are not limited to, for example brassiere wires, and functional garments having water repellent or waterproof properties.

The industrial materials may include, but are not limited to, for example fastening members, automatic switching devices, temperature-sensitive sensors, and power conversion equipment.

However, the above-mentioned examples of application are only illustrative, and therefore a variety of other uses can be considered. In addition, such uses are not particularly limited so long as shape memory effects of PHA are utilized.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view of morphological changes showing rubber-elasticity and shape-memory effects of a PHBV film prepared in Example 2 of the present invention;

FIGS. 2a to 2d are photographs showing a shape (b) in which a PHBV strip (a) prepared in Example 2 of the present invention is permanently deformed into a coil-shape, a shape (c) in which the coil-shape of the PHBV strip is stretched and temporarily shaped, and a shape (d) in which the PHBV strip having a temporarily shape is heated and recovered to its original coil-shape, respectively;

FIG. 3 is a view showing construction of a plasmid in Example 3 of the present invention;

FIG. 4 is a restriction map of a phb locus (gene for biosynthesis of a short-chain-length PHA) in Pseudomonas sp. HJ-2 in Example 3 of the present invention; and

FIGS. 5 and 6 are amino acid sequences of a phb locus (gene for biosynthesis of short-chain-length PHA) in Pseudomonas sp. HJ-2.

### DETAILED DESCRIPTION OF PREFERRED EMBODIMENTS

Now, the present invention will be described in more detail with reference to the following examples. These examples are provided only for illustrating the present invention and should not be construed as limiting the scope and sprit of the present invention.

### EXAMPLES

### Example 1

### Preparation of poly(3HB-co-3HV) Block Copolymer Film Having Shape Memory Effects

Poly(3HB-co-3HV) block copolymers were biosynthesized by culturing Pseudomonas sp. HJ-2 at pH 7 using heptanoic acid as a sole carbon source. The culture was crushed to extract the poly(3HB-co-3HV) block copolymers, and the crude extracts were purified with methanol and hexane. Upon analyzing the purified poly(3HB-co-3HV) block copolymers, a variety of block copolymers were obtained which contained 20 to 70 mol % of 3-hydroxyvalerate (3HV) depending upon experimental conditions such as culturing conditions and the like. In addition, it was confirmed that all of the thus-obtained block copolymers exhibit similar degrees of shape memory effects.

For reference, blending between the above-mentioned block copolymers and third PHAs or polymers was also prepared. In this case, the blending also exhibited shape memory effects, although there were differences from one another to a certain degree.

20% by weight of the thus-obtained poly(3HB-co-3HV) block copolymer (containing 35 mol % of 3-hydroxyvalerate (3HV)) was added to chloroform and poured into a Teflon dish, thereby preparing a poly(3HB-co-3HV) block copolymer film (a PHBV film) having a thickness of about 0.2 mm.

### Example 2

### Physical Properties of poly(3HB-co-3HV) Block Copolymer Film

In order to confirm the lowest temperature at which an existing hard segment is removed, a PHBV film obtained in Example 1 was stretched at different temperatures and held at the stretched state for about 30 seconds, followed by exposure to 90° C. For example, two water baths at 60° C. and 90° C. are consecutively prepared, and the PHBV film was stretched in the water bath at 60° C. and held for about one minute. Immediately thereafter, the stretched PHBV film was transferred to the water bath at 90° C. so as to confirm on whether the film is contracted or not. The lowest temperature at which the stretched PHBV film is not contracted upon exposure thereof to 90° C. can be determined as the lowest temperature at which the existing hard segment is removed. In repeated experiments, the PHBV films were subjected to uniaxial orientation at an elongation percentage of 600% and were held at room temperature for about one minute, thereby preparing deformed samples. The deformed samples were exposed to vapor having different temperatures and were cooled to room temperature to determine lengths thereof. The recovered samples were annealed at room temperature for about 3 minutes before they were used in subsequent repetitive experiments. 5 repeated experiments were carried out for three films, and the results thus obtained were averaged.

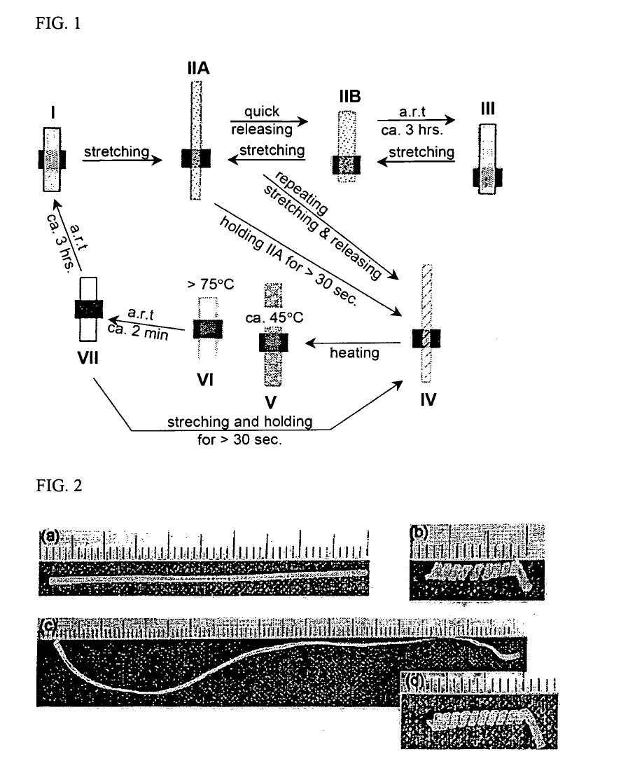

The experimental results showed that the PHBV films have leathery properties and returns to the form of film under various conditions, as shown in FIG. 1. When the leathery film (I) is stretched for a short period of time, a rubber-elastic film (II) having a maximum elongation percentage of about 700% is obtained. The film (II) turns into a leathery film (III) when it is annealed at room temperature for several hours, and the leathery film (III) becomes about 10% longer than the film (I). When it is stretched again, the leathery film (III) becomes a rubber-elastic film (II). When the film (II) is subjected to repetitive stretching and releasing processes, or the film (IIA) is held in the stretched state for more than 30 seconds, an oriented leathery film (IV) is obtained. Surprisingly, upon heating the film (IV), it contracts and returns to its original state.

A permanent shape is made by melting crystallites of the polymers to a temperature higher than 95° C. and annealing the melted polymers at room temperature, thereby inducing crystallization thereof into a permanent shape. The PHBV polymers are significantly decomposed above 150° C. The polymer sample having the permanent shape corresponds to the film (I) as shown in FIG. 1. A temporary shape is made by stretching the polymer sample to 600% and holding it at that state for more than 30 seconds. It is surmised that, during stretching and holding the sample, domains having new arrangement are formed, thereby leading to a temporary shape. The sample having the temporary shape corresponds to a film (IV) in FIG. 1. The sample having the temporary shape recovers its original shape upon heating (see V, VI and VII of FIG. 1). Initial shrinkage was observed at 45° C., and shrinkage substantially stopped at about 75° C.

Referring to FIG. 2, a sample (b) having a permanent coil-shape was prepared by winding a strip (a) into a coil-shape, heating it at 110° C. for 10 minutes, and annealing the heated coil strip at room temperature for 10 minutes. A strip (c) having a temporary shape was prepared by stretching the coil strip (b) at room temperature by hands. When the deformed strip (c) was exposed to vapor at 80° C., the temporary shape has returned to its original coil-shaped sample (d).

### Example 3

### Cloning of Short-Chain-Length PHA Synthetic (phb) Locus

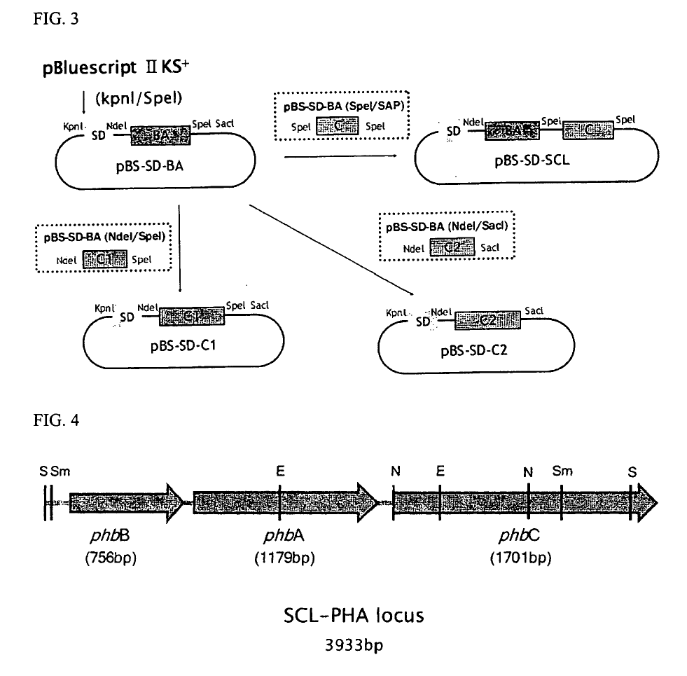

In order to clone a short-chain-length PHA synthetic gene of Pseudomonas sp. HJ-2, short-chain-length PHA synthetic genes of strains capable of synthesizing other short-chain-length PHAs were aligned to thereby prepare primers choi3 and choi4 on the basis of conserved regions. Upon performing PCR using these primers, a 0.6-kb PCR product was obtained and then cloned into a T-vector (pGEM-SCL). A base sequence of pGEM-SCL was sequenced, and Blast X search was performed. As a result, pGEM-SCL showed 75% amino acid sequence homology with PHB synthase of Pseudomonas sp. 61-3. The 0.6-kb PCR product was DIG-labeled to use as a probe for cloning short-chain-length PHA synthase. The total genomic DNA of Pseudomonas sp. HJ-2 was extracted and cleaved with various restriction enzymes, and Southern hybridization was carried out with a DIG-labeled probe using a DIG diction kit. As a result, positive signals were respectively appeared at about 4 kb, 1.5 kb, 1.2 kb, 3.5 kb and 1.6 kb, and 0.6 kb fragments, when the genomic DNA was cleaved with respective restriction enzymes Sac I, EcoR I, Nco I, Sma I and Pst I. Among restriction enzymes used herein, Nco I and Sma I are also present in DNA which was used as the probe, and based on this fact, it was possible to plot an approximate restriction map. On the basis of the restriction map, a partial genomic library was constructed using 4-kb DNA, which was obtained by cleaving the total genomic DNA of Pseudomonas sp. HJ-2 with Sac I, and a pBluescript II KS⁺ vector. Using colony hybridization, the partial genomic library containing a 4-kb Sac I fragment was subjected to clone screening (pBS-S53). Using restriction enzymes and PCR, it was re-confirmed that pBS-S53 is a desired positive clone. Upon analyzing base sequences of these clones, it could be seen that about 100 bp, corresponding to a C-terminal part of a synthase gene, was lacking. For the remaining parts, other synthase genes were aligned to construct primers HJ-2-PHB-N and HJ-2-PHB-C which were then subjected to PCR, thereby obtaining a 0.8-kb PCR product. The thus-obtained 0.8-kb PCR product was cloned into pDrive vector (pD-SCL). From the results of DNA sequencing of the pD-SCL clone thus obtained, it could be seen that the thus-obtained construct is a C-terminal of the PHB synthase. A restriction map of a phb locus is disclosed in FIG. 3.

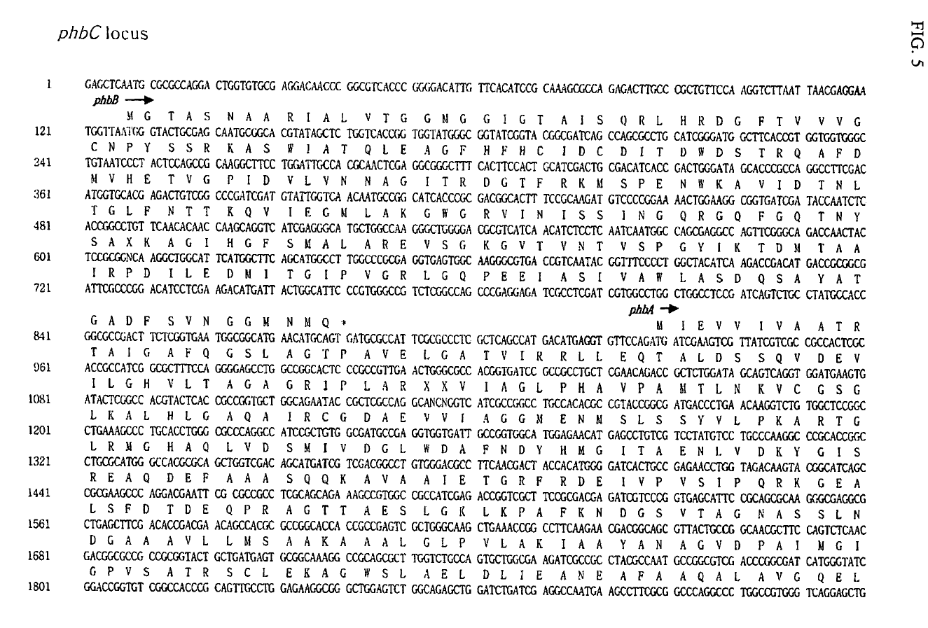

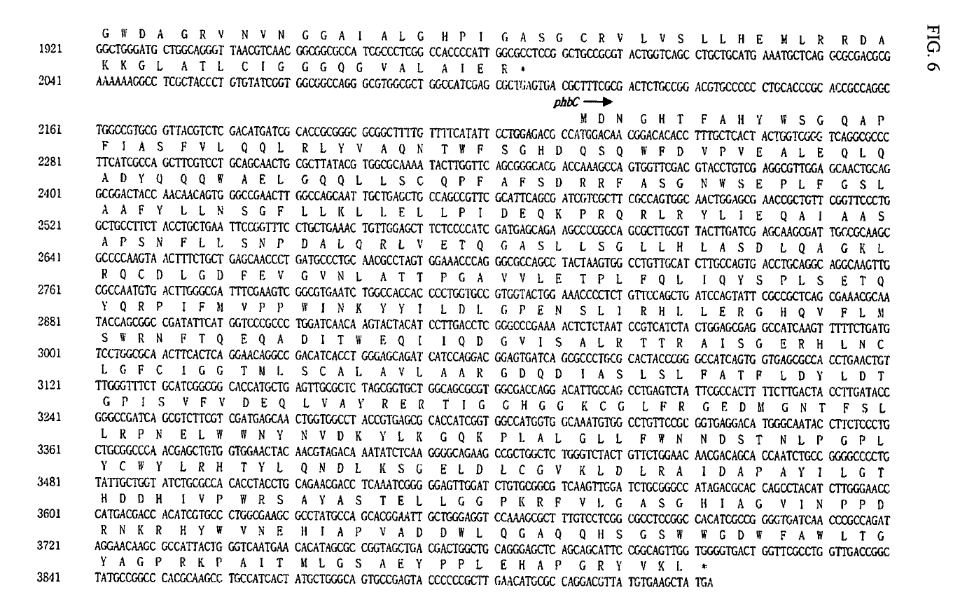

From complete interpretation of base sequences of both pBS-S53 and pD-SCL and analysis using vector NT1 (InforMax, Inc.), it could be seen that the resulting construct is a phb locus and there are three open reading frames (ORFs) (see FIG. 4). ORF1 is NADPH-dependent acetoacetyl-CoA reductase (PhbB_{HJ-2}), and consists of 765 bp, 255 amino acids (see SEQ ID NO: 12) and exhibits 69% amino acid sequence homology with PhbB of Pseudomonas sp. 61-3. ORF2 is β-ketothiolase (PhbA_{HJ-2}), and consists of 1179 bp, 393 amino acids (see SEQ ID NO: 12) and exhibits 72% amino acid sequence homology with PhbA of Pseudomonas aeruginosa. ORF3 encodes PHB synthase (PhbC_{HJ-2}), and consists of 1701 bp, 567 amino acids (see SEQ ID NO: 12) and exhibits 69% amino acid sequence homology with Pseudomonas sp. 61-3. Similar to strains synthesizing other short-chain-length PHAs, genes involved in biosynthesis of the short-chain-length PHA of HJ-2 forms 1 operon (phbBAC_{HJ-2}). However, this operon has a different composition than that of a representative strain containing a short-chain-length PHA synthase, Wautersia eutropha (formerly known as Ralstonia eutropha), and has the same composition as that of Pseudomonas sp. 61-3, known to contain both short-chain-length PHA and medium-chain-length PHA synthase genes. For the amino acid sequence of the short-chain-length PHA synthetic (phb) locus of Pseudomonas sp. HJ-2, reference is made to FIGS. 5 and 6.

Lipase box-like sequence is a highly conservative sequence of polyester synthase, and the active site residue, cysteine, located within the lipase box-like sequence, is known as the region where transesterification reaction occurs. In PhbCRe of a representative strain, W. eutropha, it was reported that cysteine, an amino acid at position 319, is known to be responsible for transesterification. In PhbC of Pseudomonas sp. HJ-2, it is believed that the 300^{th} amino acid residue, cysteine is a site where the transesterification reaction take places. In other PHA synthases, cysteine, aspartic acid and histidine, which form a catalytic triad, are all present as amino acids at positions 300, 459 and 489. Shine-Dalgarno (SD) sequence (AGGA box), known as the ribosome-binding site (RBS), could be found in 10-bp upstream of ATG which is an initiation codon of phbB, phbA and phbC genes.

Plasmids and PCR primers utilized in this example are summarized in Tables 1 and 2 as shown below.

TABLE 1

| Plasmids | Characteristics |
|---|---|
| pBluescript II KS⁺ | Ap^{r} lacPOZT7 and T3 promoter |
| pGEM T-easy vector | Ap^{r} lacPOZ T7 and SP6 promoter |
| pDrive vector | Ap^{r} Km^{r} lacPOZ T7 and SP6 promoter |
| pGEM-SCL | 0.6 kb PCR product of phbC_{HJ-2} in pGEM T-easy |
| | vector |
| pBS-S53 | 3.8 kb Sac I fragment of phbC_{HJ-2} in pBluescript |
| | II KS(+) |
| pD-SCL | 0.8 kb PCR product of phbC_{HJ-2} in pDrive vector |
| pBS-SCL | pBluescript II KS(+) derivative: phbBAC_{HJ-2} |
| pD-SCL | pDrive vector derivative phbC_{HJ-2} |
| pBS-SD-C1 | pBluescript II KS(+) derivative: phaC1_{HJ-2} |
| pBS-SD-C2 | pBluescript II KS(+) derivative; phaC2_{HJ-2} |

TABLE 2

| | |
|---|---|
| Oligonucleotide | Sequences |

| | |
|---|---|
| Choi3 | 5′-CCGCCSTGSATCAAGTAC-3′ |
| Choi4 | 5′-GYTSGTGSYGTCYYCGTTCC-3′ |
| HJ-PHB-N | 5′-CACCATGCTGAGTTGCGCTCTAGC-3′ |
| HJ-PHB-C | 5′-TCADMSYTTYACRTARCGKCCTGGYGC-3′ |
| SCL-1 | 5′-GATCGATACCAATCTCACCG-3′ |
| SCL-2 | 5′-CAAAGCCAGTGGTTCGACGTA-3′ |
| SCL-3 | 5′-CTGCTGAAACTGTTGGAGC-3′ |
| SD-BA-N | 5′-GGGGGTACCAATAAGGAGATATACATATGG |
| | GTACTGCGAGCAATGCG-3′ |
| BA-C | 5′-CCCACTAGTTCAGCGCTCGATGGCCAGC- |
| | 3′ |
| SD-phbC-N | 5′-GGGCATATGACCCAGAAGAACAACAGCG- |
| | 3′ |
| phbC-C | 5′-CCCACTAGTTCADMSCTTYACRTAACGTCC |
| | TGGCGCYGC-3′ |

### INDUSTRIAL APPLICABILITY

As apparent from the above description, a PHA block copolymer having a particular composition in accordance with the present invention exhibits orientation-induced rubber-elasticity and shape memory effects with a fast shape-recovery rate, and therefore such characteristics in combination with physical properties such as biodegradability and biocompatibility unique to PHA enable application thereof to a variety of uses.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A PHA block copolymer having orientation-induced rubber-elasticity and temperature-sensitive shape memory effects, comprising:
a plurality of 3-hydroxybutyrate (3HB) blocks of Formula 1 as a repeating unit: 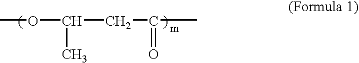
wherein m is not less than 2; and
a plurality of 3-hydroxyvalerate (3HV) blocks of Formula 2 as a repeating unit: 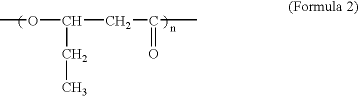
wherein n is not less than 2.

2. The PHA block copolymer according to claim 1, wherein the block copolymer is heated to a temperature ranging from a melting point to thermal decomposition temperature thereof, thereby preparing a permanently deformed particular shape, and the permanently shaped material is subjected to constant external force at near room temperature for a predetermined period of time, thereby forming a shaped material having a temporary shape.

3. The PHA block copolymer according to claim 2, wherein the temporarily shaped material is rapidly recovered to its original state of the permanently shaped material by heating the temporarily shaped material to a temperature ranging from a glass transition temperature to melting point thereof.

4. The PHA block copolymer according to claim 1, wherein the content of 3HV in the total monomers of the copolymer is within the range of 10 to 90 mol %.

5. The PHA block copolymer according to claim 1, wherein the molecular weight of the copolymer is approximately in the range of several tens of thousands to several millions.

6. The PHA block copolymer according to claim 1, wherein the copolymer further comprises not more than 70 mol % of a hydroxy acid repeating group of Formula 3, based on the total polymer: 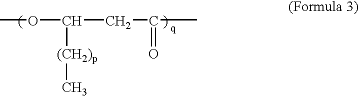
wherein p and q are independently not less than 2.

7. A method for preparing a PHA block copolymer of claim 1 by chemical synthesis, or biosynthesis using microorganisms.

8. The method according to claim 7, wherein the PHA block copolymer is prepared using saturated and/or unsaturated carboxylic acid as a carbon source and a Pseudomonas sp. HJ-2 strain (Accession No. KCTC 0406 BP).

9. The method according to claim 8, wherein the PHA block copolymer is prepared by culturing the Pseudomonas sp. HJ-2 strain with supply of heptanoic acid as a sole carbon source.

10. A blending or composite having temperature-sensitive shape memory effects, comprising a block copolymer of claim 1 and one or more third polymers.

11. A short-chain-length PHA synthetic gene of a Pseudomonas sp. HJ-2 strain capable of biosynthesizing a PHA block copolymer of claim 1.

12. The short-chain-length PHA synthetic gene according to claim 11, wherein the gene includes a gene having a sequence as set forth in SEQ. ID. NO: 12.

13. The short-chain-length PHA synthetic gene according to claim 11, wherein the gene includes a gene having a sequence as set forth in SEQ. ID. NO: 13.

14. The short-chain-length PHA synthetic gene according to claim 11, wherein the gene includes a gene having a sequence as set forth in SEQ. ID. NO: 14.

15. A method for preparing a PHA block copolymer of claim 1, by culturing a microorganism transformed with a short-chain-length PHA synthetic gene of a Pseudomonas sp. HJ-2 strain capable of biosynthesizing a PHA block copolymer of claim 1 or by cell-free protein synthesis using the same gene.

16. A method of using shape memory effects of a PHA block copolymer of claim 1.

17. The method according to claim 16, wherein the PHA block copolymer is used as a medical material, a material for living necessaries, a fiber/fabric material or an industrial material.

18. The method according to claim 17, wherein the medical material is used in an angioplasty stent, an implant tube for the urethrae and the esophagi, a device for vascular anastomosis, a dental implant, or an orthodontic spring or wire.

19. A blending or composite having temperature-sensitive shape memory effects, comprising a block copolymer of claim 6 and one or more third polymers.


SEQUENCE LISTING (14 sequences)

SEQ ID NO: 1 - DNA, 18 bp, Artificial Sequence
  Primer
ccgccstgsatcaagtac

SEQ ID NO: 2 - DNA, 20 bp, Artificial Sequence
  Primer
gytsgtgsygtcyycgttcc

SEQ ID NO: 3 - DNA, 24 bp, Artificial Sequence
  Primer
caccatgctgagttgcgctctagc

SEQ ID NO: 4 - DNA, 27 bp, Artificial Sequence
  Primer
tcadmsyttyacrtarcgkcctggygc

SEQ ID NO: 5 - DNA, 20 bp, Artificial Sequence
  Primer
gatcgataccaatctcaccg

SEQ ID NO: 6 - DNA, 21 bp, Artificial Sequence
  Primer
caaagccagtggttcgacgta

SEQ ID NO: 7 - DNA, 19 bp, Artificial Sequence
  Primer
ctgctgaaactgttggagc

SEQ ID NO: 8 - DNA, 47 bp, Artificial Sequence
  Primer
gggggtaccaataaggagatatacatatgggtactgcgagcaatgcg

SEQ ID NO: 9 - DNA, 28 bp, Artificial Sequence
  Primer
cccactagttcagcgctcgatggccagc

SEQ ID NO: 10 - DNA, 28 bp, Artificial Sequence
  Primer
gggcatatgacccagaagaacaacagcg

SEQ ID NO: 11 - DNA, 39 bp, Artificial Sequence
  Primer
cccactagttcadmscttyacrtaacgtcctggcgcygc

SEQ ID NO: 12 - DNA, 756 bp, Pseudomonas sp. HJ-2
  variation (482): n=A, C, G or T
atgggtactgcgagcaatgcggcacgtatagctctggtcaccggtggtatgggcggtatcggtacggcgatcagccagcgcctgcatcgggatggcttcaccgtggtggtgggctgtaatccctactccagccgcaaggcttcctggattgccacgcaactcgaggcgggctttcacttccactgcatcgactgcgacatcaccgactgggatagcacccgccaggccttcgacatggtgcacgagactgtcggcccgatcgatgtattggtcaacaatgccggcatcacccgcgacggcactttccgcaagatgtccccggaaaactggaaggcggtgatcgataccaatctcaccggcctgttcaacacaaccaagcaggtcatcgagggcatgctggccaagggctggggacgcgtcatcaacatctcctcaatcaatggccagcgaggccagttcgggcagaccaactactccgcggncaaggctggcattcatggcttcagcatggccttggcccgcgaggtgagtggcaagggcgtgaccgtcaatacggtttcccctggctacatcaagaccgacatgaccgcggcgattcgcccggacatcctcgaagacatgattactggcattcccgtgggccgtctcggccagcccgaggagatcgcctcgatcgtggcctggctggcctccgatcagtctgcctatgccaccggcgccgacttctcggtgaatggcggcatgaacatgcagtga

SEQ ID NO: 13 - DNA, 1179 bp, Pseudomonas sp. HJ-2
  variation (207): n=A, C, G or T
atgatcgaagtcgttatcgtcgccgccactcgcaccgccatcggcgctttccaggggagcctggccggcactcccgccgttgaactgggcgccacggtgatccgccgcctgctcgaacagaccgctctggatagcagtcaggtggatgaagtgatactcggccacgtactcaccgccggtgctggcagaataccgctcgccaggcancnggtcatcgccggcctgccacacgccgtaccggcgatgaccctgaacaaggtctgtggctccggcctgaaagccctgcacctgggcgcccaggccatccgctgtggcgatgccgaggtggtgattgccggtggcatggagaacatgagcctgtcgtcctatgtcctgcccaaggcccgcaccggcctgcgcatgggccacgcgcagctggtcgacagcatgatcgtcgacggcctgtgggacgccttcaacgactaccacatggggatcactgccgagaacctggtagacaagtacggcatcagccgcgaagcccaggacgaattcgccgccgcctcgcagcagaaagccgtggccgccatcgagaccggtcgcttccgcgacgagatcgtcccggtgagcattccgcagcgcaagggcgaggcgctgagcttcgacaccgacgaacagccacgcgccggcaccaccgccgagtcgctgggcaagctgaaaccggccttcaagaacgacggcagcgttactgccggcaacgcttccagtctcaacgacggcgccgccgcggtactgctgatgagtgcggcaaaggccgcagcgcttggtctgccagtgctggcgaagatcgccgcctacgccaatgccggcgtcgacccggcgatcatgggtatcggaccggtgtcggccacccgcagttgcctggagaaggcgggctggagtctggcagagctggatctgatcgaggccaatgaagccttcgcggcccaggccctggccgtgggtcaggagctgggctgggatgctggcagggttaacgtcaacggcggcgccatcgccctcggccaccccattggcgcctccggctgccgcgtactggtcagcctgctgcatgaaatgctcaggcgcgacgcgaaaaaaggcctcgctaccctgtgtatcggtggcggccagggcgtggcgctggccatcgagcgctga

SEQ ID NO: 14 - DNA, 1701 bp, Pseudomonas sp. HJ-2
atggacaacggacacacctttgctcactactggtcgggtcaggcgcccttcatcgccagcttcgtcctgcagcaactgcgcttatacgtggcgcaaaatacttggttcagcgggcacgaccaaagccagtggttcgacgtacctgtcgaggcgttggagcaactgcaggcggactaccaacaacagtgggccgaacttggccagcaattgctgagctgccagccgttcgcattcagcgatcgtcgcttcgccagtggcaactggagcgaaccgctgttcggttccctggctgccttctacctgctgaattccggtttcctgctgaaactgttggagcttctccccatcgatgagcagaagccccgccagcgcttgcgttacttgatcgagcaagcgattgccgcaagcgccccaagtaactttctgctgagcaaccctgatgccctgcaacgcctagtggaaacccagggcgccagcctactaagtggcctgttgcatcttgccagtgacctgcaggcaggcaagttgcgccaatgtgacttgggcgatttcgaagtcggcgtgaatctggccaccacccctggtgccgtggtactggaaacccctctgttccagctgatccagtattcgccgctcagcgaaacgcaataccagcggccgatattcatggtcccgccctggatcaacaagtactacatccttgacctcgggcccgaaaactctctaatccgtcatctactggagcgaggccatcaagtttttctgatgtcctggcgcaacttcactcaggaacaggccgacatcacctgggagcagatcatccaggacggagtgatcagcgccctgcgcactacccgggccatcagtggtgagcgccacctgaactgtttgggtttctgcatcggcggcaccatgctgagttgcgctctagcggtgctggcagcgcgtggcgaccaggacattgccagcctgagtctattcgccacttttcttgactaccttgataccgggccgatcagcgtcttcgtcgatgagcaactggtggcctaccgtgagcgcaccatcggtggccatggtggcaaatgtggcctgttccgcggtgaggacatgggcaataccttctccctgctgcggcccaacgagctgtggtggaactacaacgtagacaaatatctcaaggggcagaagccgctggctctgggtctactgttctggaacaacgacagcaccaatctgccggggcccctgtattgctggtatctgcgccacacctacctgcagaacgacctcaaatcgggggagttggatctgtgcggcgtcaagttggatctgcgggccatagacgcaccagcctacatcttgggaacccatgacgaccacatcgtgccctggcgaagcgcctatgccagcacggaattgctgggaggtccaaagcgctttgtcctcggcgcctccggccacatcgccggggtgatcaacccgccagataggaacaagcgccattactgggtcaatgaacacatagcgccggtagctgacgactggctgcagggagctcagcagcattccggcagttggtggggtgactggttcgcctggttgaccggctatgccggcccacgcaagcctgccatcactatgctgggcagtgccgagtaccccccgcttgaacatgcgccaggacgttatgtgaagctatga